(19) United States
(12) Patent Application Publication
(10) Pub. No.: US 2024/0225623 A1
(43) Pub. Date: Jul. 11, 2024

(54) TISSUE SAMPLE DEVICE AND METHODS

(71) Applicant: Boston Scientific Scimed, Inc., Maple Grove, MN (US)

(72) Inventors: James Weldon, Newton, MA (US); Paul Smith, Smithfield, RI (US); Madinah Shamsu, Antioch, TN (US); Andrew James Schaubhut, Bolton, MA (US); Jeffrey V. Bean, Fitchburg, MA (US)

(73) Assignee: Boston Scientific Scimed, Inc., Maple Grove, MN (US)

(21) Appl. No.: 18/409,355

(22) Filed: Jan. 10, 2024

Related U.S. Application Data
(60) Provisional application No. 63/438,384, filed on Jan. 11, 2023.

(51) Int. Cl. A61B 10/04

(52) U.S. Cl. CPC A61B 10/04; A61B 2010/0208

(57) ABSTRACT

A tissue collection system for collecting a sample from the body. The tissue collection system may comprise a tubular member having a proximal end region and a distal end region and defining a lumen extending from the proximal end region to the distal end region, an elongate shaft slidably disposed within the lumen of the tubular member, the elongate shaft extending from a proximal end region to a distal end region, and a tissue collection member disposed adjacent to the distal end region. The tissue collection member may comprise a helically wound coil including a plurality of windings.

### CROSS-REFERENCE TO RELATED APPLICATIONS

This application claims the benefit of priority under 35 U.S.C. §119 of U.S. Provisional Application No. 63/438,384, filed Jan. 11, 2023, the entire disclosure of which is hereby incorporated by reference.

### TECHNOLOGY FIELD

The present application relates generally to medical devices for tissue sample collection, and more particularly, to medical devices for tissue sample collection resulting in greater cellular or other tissue yield.

### BACKGROUND

Certain medical tests require sampling of cells from target areas of a subject's body. For instance, a screening test for detecting potentially pre-cancerous and cancerous tissues in a subject's body may include taking samples of tissue or cells from a target area of the subject's body. A tissue collection device may be used to collect cells or other tissues from the target area. Tissue collection from some parts of the anatomy may be difficult. There is an ongoing need to provide alternative medical devices as well as alternative methods for manufacturing and using medical devices.

### SUMMARY

This disclosure provides design, material, manufacturing methods, and use alternatives for medical devices.

In a first example, a tissue collection system may comprise a tubular member having a proximal end region and a distal end region and defining a lumen extending from the proximal end region to the distal end region, an elongate shaft slidably disposed within the lumen of the tubular member, the elongate shaft extending from a proximal end region to a distal end region, and a tissue collection member disposed adjacent to the distal end region. The tissue collection member may comprise a helically wound coil including a plurality of windings.

Alternatively or additionally to any of the examples above, in another example, the tissue collection member may be coupled to the elongate shaft.

Alternatively or additionally to any of the examples above, in another example, the tissue collection member may be coupled to the elongate shaft along less than an entire length of the tissue collection member.

Alternatively or additionally to any of the examples above, in another example, the tissue collection system may further comprise a pull wire coupled to the helically wound coil.

Alternatively or additionally to any of the examples above, in another example, the pull wire may be configured to be actuated to adjust a pitch of the helically wound coil.

Alternatively or additionally to any of the examples above, in another example, an inner surface of the helically wound coil may contact an outer surface of the elongate shaft.

Alternatively or additionally to any of the examples above, in another example, the helically wound coil may be formed from a ribbon having a first lateral side and a second lateral side.

Alternatively or additionally to any of the examples above, in another example, a first lateral side of the ribbon may be adjacent to the elongate shaft and the second lateral side of the ribbon may be radially spaced from the elongate shaft.

Alternatively or additionally to any of the examples above, in another example, the tissue collection system may further comprise a plurality of cavities, the plurality of cavities may be defined by a space between an inner surface of the ribbon and the elongate shaft.

Alternatively or additionally to any of the examples above, in another example, the plurality of cavities may each have an opening.

Alternatively or additionally to any of the examples above, in another example, each opening of the plurality of cavities may be distal facing.

Alternatively or additionally to any of the examples above, in another example, each opening of the plurality of cavities may be proximal facing.

Alternatively or additionally to any of the examples above, in another example, a maximum outer diameter of the tissue collection member may reduce in diameter in a distal direction.

In another example, a tissue collection system may comprise a tubular member having a proximal end region and a distal end region and defining a lumen extending from the proximal end region to the distal end region, an elongate shaft slidably disposed within the lumen of the tubular member, the elongate shaft extending from a proximal end region to a distal end region, and a tissue collection member disposed adjacent to the distal end region. The tissue collection member may comprise a plurality of scraping members having an open first end and a second end coupled to the elongate shaft.

Alternatively or additionally to any of the examples above, in another example, each scraping member of the plurality of scraping members may define a cavity therein.

In another example, a tissue collection system may comprise a tubular member having a proximal end region and a distal end region and defining a lumen extending from the proximal end region to the distal end region, an elongate shaft slidably disposed within the lumen of the tubular member, the elongate shaft extending from a proximal end region to a distal end region, and a tissue collection member disposed adjacent to the distal end region. The tissue collection member may comprise a helically wound coil including a plurality of windings and a plurality of recesses between adjacent windings of the plurality of windings.

Alternatively or additionally to any of the examples above, in another example, the tissue collection member may be coupled to the elongate shaft.

Alternatively or additionally to any of the examples above, in another example, the tissue collection member may be coupled to the elongate shaft along less than an entire length of the tissue collection member.

Alternatively or additionally to any of the examples above, in another example, the tissue collection system may further comprise a pull wire coupled to the helically wound coil.

Alternatively or additionally to any of the examples above, in another example, the pull wire may be configured to be actuated to adjust a pitch of the helically wound coil.

Alternatively or additionally to any of the examples above, in another example, an inner surface of the helically wound coil may contact an outer surface of the elongate shaft.

Alternatively or additionally to any of the examples above, in another example, the helically wound coil may be formed from a filament having a thickness.

Alternatively or additionally to any of the examples above, in another example, the thickness of the filament may reduce from a proximal end to a distal end thereof.

In another example, a tissue collection system may comprise a tubular member having a proximal end region and a distal end region and defining a lumen extending from the proximal end region to the distal end region, an elongate shaft slidably disposed within the lumen of the tubular member, the elongate shaft extending from a proximal end region to a distal end region, and a tissue collection member disposed adjacent to the distal end region. The tissue collection member may comprise a ribbon having a proximal end, a distal end, a first lateral side extending between the proximal end and the distal end, and a second lateral side extending between the proximal end and the distal end. The ribbon may be wound about the elongate shaft to form a helically wound coil including a plurality of windings.

Alternatively or additionally to any of the examples above, in another example, the first lateral side of the ribbon may be adjacent to the elongate shaft and the second lateral side of the ribbon may be radially spaced from the elongate shaft.

Alternatively or additionally to any of the examples above, in another example, the tissue collection system may further comprise a plurality of cavities. The plurality of cavities may be defined by a space between an inner surface of the ribbon and the elongate shaft.

Alternatively or additionally to any of the examples above, in another example, the plurality of cavities may each have an opening.

Alternatively or additionally to any of the examples above, in another example, each opening of the plurality of cavities may be distal facing.

Alternatively or additionally to any of the examples above, in another example, each opening of the plurality of cavities may be proximal facing.

Alternatively or additionally to any of the examples above, in another example, a maximum outer diameter of the tissue collection member may reduce in diameter in a distal direction.

In another example, a tissue collection system may comprise a tubular member having a proximal end region and a distal end region and defining a lumen extending from the proximal end region to the distal end region, an elongate shaft slidably disposed within the lumen of the tubular member, the elongate shaft extending from a proximal end region to a distal end region, and a tissue collection member disposed adjacent to the distal end region. The tissue collection member may comprise a plurality of scraping members having an open first end and a second end coupled to the elongate shaft.

Alternatively or additionally to any of the examples above, in another example, each scraping member of the plurality of scraping members may define a cavity therein.

Alternatively or additionally to any of the examples above, in another example, the open first end of the plurality of scraping members may be distally facing.

Alternatively or additionally to any of the examples above, in another example, the open first end of the plurality of scraping members may be proximally facing.

Alternatively or additionally to any of the examples above, in another example, a maximum outer diameter of the tissue collection member may reduce in diameter in a distal direction.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 illustrates a partial cross-sectional side view of an illustrative tissue collection device system for delivering a tissue collection device to a target region in a use configuration within a vessel;

FIG. 2 illustrates a perspective view of the distal end region of the tissue collection device of FIG. 1;

FIG. 3 illustrates a perspective view of distal end region of another illustrative tissue collection device; and

FIG. 4 illustrates a perspective view of distal end region of another illustrative tissue collection device.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION OF EXEMPLARY EMBODIMENTS

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements but may include other elements not expressly listed or inherent to such process, method, article, or apparatus. Additionally, terms that indicate the geometric shape of a component/surface refer to exact and approximate shapes.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Endoscopic retrograde cholangiopancreatography (ERCP) is a procedure that utilizes both endoscopic and fluoroscopic techniques to diagnose and treat issues arising in the common bile duct (CBD) and pancreatic ducts (PD). One of the main issues being treated today is strictures in the CBD from such as, but not limited to, primary sclerosing cholangitis (PSC), cancer of the bile duct, damage and scarring due to a gallstone in the bile duct, etc. However, biliary structures may have a low cancer sensitivity rate which may lead to a false-negative diagnosis during an ERCP procedure. In some cases, low sensitivity may be linked to inadequate tissue sampling which is a primary limiting factor of detecting potential malignancy. When a clinician needs to take a sample of the stricture, one of the most common ways to do this is by using a cytology brush. However, the sensitivity of a cytology brush may be between about 30-60%. The sensitivity of cytology brushes may be mainly linked to insufficient quantities of samples. One of the reasons cytology brushes may not be able to collect enough samples is the use of soft bristles. Another reason may be losing samples during the procedure.

What may be desirable is a sample collection device that collects a sufficient amount of sample for better prediction of biliary cancer. While the present disclosure is described with respect to the common bile duct and pancreatic ducts, the devices and methods are not limited to such use. For example, the devices and methods described herein may be used in any portion of the anatomy, as desired. Further, the devices and methods described herein may be used either endoscopic or non-endoscopic anatomies. Some illustrative anatomies may include, but are not limited to, the mouth, the esophagus, the stomach, the duodenum, other portions of the gastrointestinal tract, the pathways leading to the lungs, other portions of the respiratory system, the urinary tract, the cervix, other reproductive anatomy, etc.

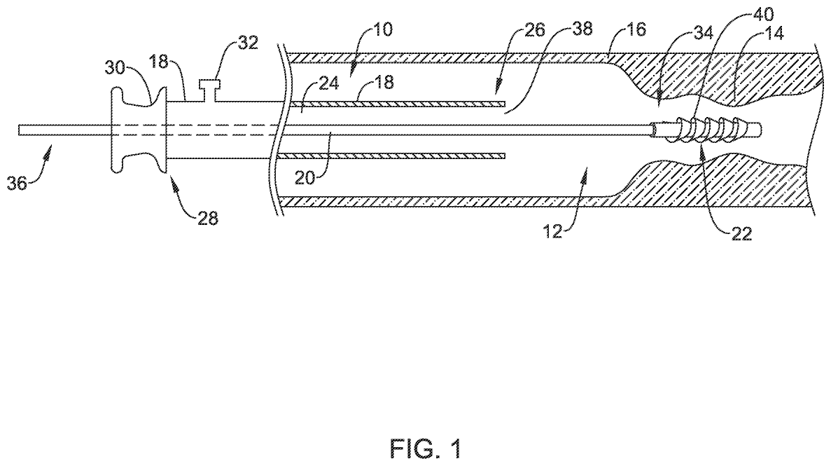

FIG. 1 is a partial cross-sectional side view of an illustrative tissue collection system 10 for delivering a tissue collection device 12 to a target region 14, such as, but not limited to, the common bile duct or pancreatic duct 16, with the tissue collection device 12 in an extended or use configuration. The tissue collection system 10 may include an outer or exterior elongate shaft or tubular member 18, such as, but not limited to, a catheter or introduction sheath. The system 10 may further include a tissue collection device 12 having an elongate shaft 20 and a tissue collection member 22 disposed on a distal portion of the elongate shaft 20. The tissue collection device 12 may be slidably disposed within a lumen 24 of the outer tubular member 18.

The outer tubular member 18 may extend proximally from a distal end region 26 to a proximal end region 28 configured to remain outside of a patient's body. A first hub or handle 30 may be coupled to the proximal end region 28 of the outer tubular member 18. In some cases, a port 32, such as an injection port, may be provided in the outer tubular member 18. Other structures to facilitate connection to other medical devices (e.g., syringe, stopcocks, Y-adapter, etc.) and to provide access to the lumen 24 may be provided. The elongate shaft 20 may extend proximally from a distal end region 34 to a proximal end region 36 configured to remain outside of a patient's body. In some cases, the handle 30 may be configured to lock or secure to the elongate shaft 20 such that the tissue collection device 12 and the outer tubular member 18 may be secured to one another at a desired relative position.

The outer tubular member 18 may include a lumen 24 extending from a distal opening 38 at the distal end region 26 to the proximal end region 28. The lumen 24 may also extend through the first handle 30. The lumen 24 of the outer tubular member 18 and the first handle 30 may be configured to slidably receive the elongate shaft 20. The lumen 24 of the outer tubular member 18 may also be configured to receive a guidewire (not explicitly shown). It is contemplated that the system 10 may be arranged such that the guidewire extends within a lumen along an entire length of the outer tubular member 18 in an “over-the-wire” manner or the guidewire may exit a side port in the outer tubular member 18 distal to the proximal end region 28 thereof in a “rapid-exchange” manner.

During insertion of the system 10 into the subject's body, or withdrawal of the system 10 from the subject's body, the tissue collection device 12 may be in a retracted position, with the elongate shaft 20 pulled proximally to position the tissue collection member 22 within the lumen 24 of the outer tubular member 18. The elongate shaft 20 of the tissue collection device 12 may be pushed distally to move the tissue collection device 12 to an extended position (shown in FIG. 1), with the tissue collection member 22 extending out of the distal opening 38, so it is exposed from the outer tubular member 18.

The elongate shaft 20 may include one or more wires twisted into an elongated form. Alternatively, the elongate shaft 20 may be a generally tubular member. In yet another example, the elongate shaft 20 may be a generally solid rod. The elongate shaft 20 may have sufficient flexibility to allow it to bend during insertion of the tissue collection device 12 into or withdrawal of the tissue collection device 12 from the subject's body. The elongate shaft 20 may have sufficient rigidity so that pushing or pulling of the elongate shaft 20 may cause extension or retraction, respectively, of the tissue collection member 22 from the outer tubular member 18. A proximal end region 36 of the elongate shaft 20 may be gripped by a user such that the user may manually push or pull the elongate shaft 20.

Dimensions of the elongate shaft 20 may vary depending upon the subject's anatomy and/or the type of procedure being performed. While not explicitly shown, the elongate shaft 20 may include a distal atraumatic tip. This distal atraumatic tip could be a rounded wire loop, flexible member, a curve flexible member, a bullet shape member, etc. An atraumatic tip may help the shaft to safely navigate to a targeted site.

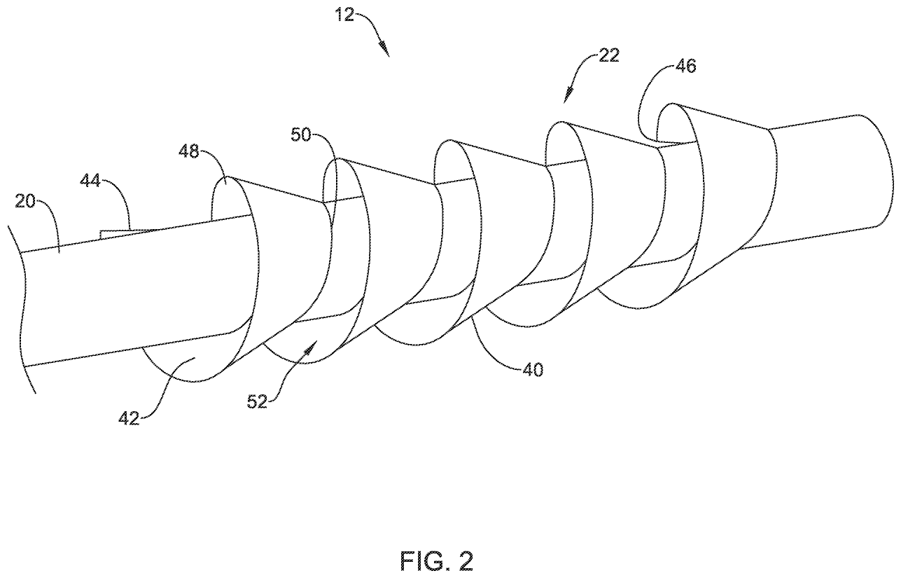

Referring additionally to FIG. 2, which illustrates a perspective view of the distal end region 34 of the tissue collection device 12, in some embodiments, the tissue collection member 22 may include a helically wound coil 40. The helically wound coil 40 may extend along a length of the elongate shaft 20 in the range of about 0.3 inches (7.62 millimeters (mm)) to about 0.55 inches (13.97 mm). The helically wound coil 40 may be formed from a ribbon 42 extending from a proximal end 44 to a distal end 46. The ribbon 42 may have a first lateral side 48 and a second lateral side 50. The first and second lateral sides 48, 50 may extend between the proximal end 44 and the distal end 46 of the ribbon.

The ribbon 42 may have a thickness that is less that a width (e.g., distance between the first lateral side 48 and the second lateral side 50) and less than a length (e.g., distance between the proximal end 44 and the distal end 46) thereof. While the ribbon 42 is shown and described as having a generally rectangular cross-sectional shape, the ribbon 42 may have other shapes, as desired, including, but not limited to, circular, oblong, polygonal, square, etc. It is contemplated that the ribbon 42 may be formed from metals, metal alloys, polymers, composites, etc.

The ribbon 42 may be helically wound about the elongate shaft 20 to form the helically wound coil 40. It is contemplated that one lateral side 48, 50 may be affixed or adjacent to the elongate shaft 20 while the opposing lateral side 48, 50 is radially spaced from the elongate shaft 20. The ribbon 42 may be coupled to the elongate shaft 20 using a number of different techniques, including, but not limited to, welding, soldering, brazing, heat bonding, adhesives, etc. It is contemplated that the coupling technique may vary depending on the material of the elongate shaft 20 and the ribbon 42. In some examples, the elongate shaft 20 and the helically wound coil 40 may be formed as a single monolithic structure. In the illustrated embodiment, the second lateral side 50 is affixed or adjacent to the elongate shaft 20 while the first lateral side 48 is radially spaced from the elongate shaft 20 to define a plurality of cavities 52 between the elongate shaft 20 and the first and second lateral sides 48, 50. Said differently, the first lateral side 48 may extend to a greater diameter than the second lateral side 50 and may be free from attachment to the elongate shaft 20. In some examples, the helically wound coil 40 may not be affixed to the elongate shaft 20 along an entire length thereof. For example, an entirety of the second lateral side 50 may not affixed to the elongate shaft 20. It is contemplated that not securing an entirety of the second lateral side 50 may allow the helically wound coil 40 to actuate to create more space between adjacent windings or compress to reduce the space between adjacent windings. In some embodiments, the helically wound coil 40 may be coupled to a pull wire. The pull wire may be proximally retracted to reduce the pitch of the adjacent windings (e.g., bring them closer together) of the helically wound coil 40. This may facilitate proximal retraction of the helically wound coil into the outer tubular member 18. Releasing the proximal force on the pull wire, if so provided, may increase the pitch of the adjacent windings (e.g., move them farther apart) of the helically wound coil 40 to allow for more tissue penetration between the windings.

The elongate shaft 20 may include any number of cavities 52 desired, such as, one, two, three, four, five, or more. It is contemplated that the number of cavities 52 may depend, at least in part, on a number of windings of the ribbon 42. The cavities 52 may be configured to trap tissue and/or cells therein. In the illustrated embodiments, the cavities 52 may have a proximally facing opening. The proximally facing openings may collect tissue during proximal retraction of the tissue collection device 12. However, this is not required. For example, in some embodiments, the first lateral side 48 may be affixed to the elongate shaft 20 while the second lateral side 50 may be radially spaced from the elongate shaft 20. Such an orientation may reverse the configuration of the cavities 52 such that the cavities have a distally facing opening. The distally facing openings may collect tissue during distal advancement of the tissue collection device 12. It is contemplated that distally facing openings may provide better contrast flow (or other fluid flow) past the tissue collection member 22 as fluid may not collect in the distally facing cavities 52 to the same extent as proximally facing cavities 52.

It is contemplated that the tissue collection member 22 may include cavities 52 having proximally facing opening, cavities 52 having a distally facing opening, or cavities 52 in which some have a proximally facing openings and some have distally facing openings. When cavities 52 are provided that have differently oriented openings, it is contemplated that the cavities 52 may be arranged in any manner desired. In one example, the proximal most cavities 52 may have proximally facing openings while the distal most cavities 52 may have distally facing openings. This is just one example.

It is contemplated that the distance between the elongate shaft 20 and the free lateral side (e.g., the first lateral side 48 in FIG. 2) may be adjusted by manipulating an angle of the width of the ribbon 42. For example, an angle of the width of the ribbon 42 (relative to a longitudinal axis of the elongate shaft 20) may be decreased (or closer to parallel) to reduce the radial profile of the tissue collection member 22 and increased (or closer to orthogonal) to increase the radial profile of the tissue collection member 22. For example, it is contemplated that the helically wound coil 40 may reduce in outer diameter in the distal direction to facilitate penetration into a tight stricture while still allowing portions of the helically wound coil 40 to contact and scrape the stricture. It is further contemplated that the pitch (e.g., distance between adjacent windings) of the helically wound coil 40 may varied to achieve a desired effect. In some cases, the helically wound coil 40 may have a uniform pitch along a length thereof. In other cases, the pitch of the helically wound coil 40 may vary or change along a length thereof. For example, the pitch of the helically would coil 40 may be modified to allow for a tight pitch in some areas and more open coil in other areas.

When the tissue collection device 12 is disposed within the outer tubular member 18, the tissue collection device 12 may be restrained in a compressed reduced diameter or delivery configuration by the outer tubular member 18 surrounding the tissue collection device 12. In the compressed configuration, the tissue collection device 12 may have a smaller diameter than the expanded deployed configuration. The distal end region 26 of the outer tubular member 18 may be positioned such that the outer tubular member 18 surrounds and covers the length of the tissue collection device 12 during delivery. The outer tubular member 18 may have sufficient hoop strength to retain the tissue collection device 12 in its reduced diameter state. However, this is not required. In some cases, the tissue collection device 12 may be free from restraint or compression when disposed within the lumen 24 of the outer tubular member 18.

To collect a sample, the tissue collection system 10 may be advanced through the body towards the target location 14, as desired. The tissue collection system 10 may be advanced with or without the use of a guidewire. The tissue collection device 12 may be deployed by actuating the proximal end region 36 of the elongate shaft 20, for example, by distally pushing the proximal end region 36, while maintaining the first handle 30 in a fixed position. Thus, the elongate shaft 20 may be distally advanced relative to the outer tubular member 18. In other words, the elongate shaft 20 may be distally advanced while the outer tubular member 18 is held stationary. The reverse configuration is also contemplated. For example, the outer tubular member 18 may be proximally retracted while the elongate shaft 20 is held stationary. As the elongate shaft 20 is distally advanced, the biasing force is removed from the exterior of the tissue collection member 22 and the tissue collection member may assume its radially expanded, unbiased, deployed configuration (if the tissue collection member 22 is compressed within the outer tubular member 18), shown in FIG. 1.

Once the tissue collection device 12 is deployed from the outer tubular member 18, the proximal end region 36 of the elongate shaft 20 may be actuated to repeatedly distally advance, proximally retract, and/or rotate the tissue collection member 22 along the target collection site 14. This may cause the tissue collection member 22 to brush or scrape against the tissue surface to dislodge and capture cells. Some cells may be trapped within the cavities 52 while others may collect on the surface of the helically wound coil 40. It is contemplated that the free lateral side (e.g., the first lateral side 48 in FIGS. 1 and 2) may have a sharp edge to scratch or dislodge a sufficient number of cells for analysis.

Once the tissue sample has been collected, the tissue collection system 10 may be removed from the body. In some examples, the elongate shaft 20 and the tissue collection member 22 may be retracted into the outer tubular member 18 for removal. This may help prevent tissue and/or cells from becoming dislodged from the tissue collection member 22 as the tissue collection device 12 is withdrawn from the body.

The elongate shaft 20 may be cut (e.g., using wire cutters or other cutting device) at a location proximal to the helically wound coil 40 such that the helically wound coil 40 may be placed into a sample container. However, this is not required. The sample may be rinsed from the helically wound coil 40 and/or the cavities 52 using a fluid, such as, but not limited to, saline. It is contemplated that collecting tissue/cells using the helically wound coil 40 and the cavities 52 may result in a larger sample size than the use of a cytology brush. This may allow the system 10 to collect a sufficient amount of sample for better prediction of biliary cancer (or other ailments).

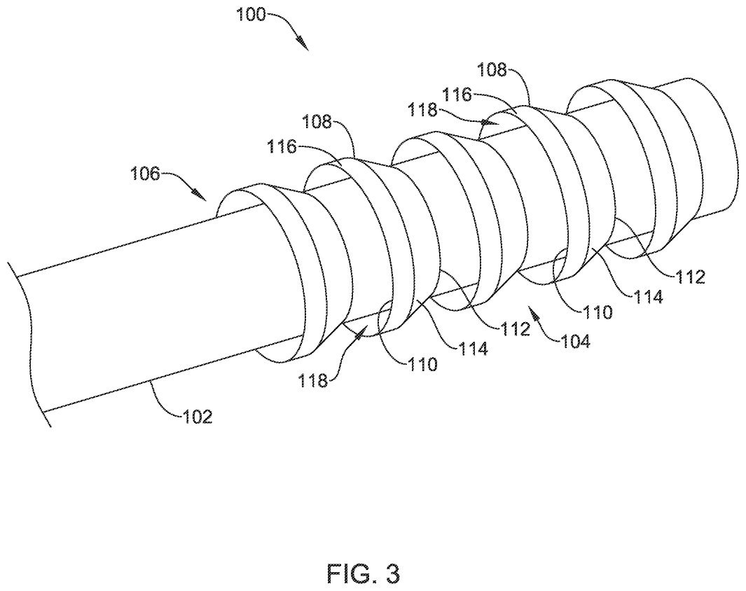

FIG. 3 is a perspective view of distal end region of another illustrative tissue collection device 100. The tissue collection device 100 may be configured to be used with a tissue collection system, such as, but not limited to the tissue collection system 10 described with respect to FIG. 1. For example, the tissue collection device 100 may be used with the system of FIG. 1 in place of tissue collection device 12. Generally, the tissue collection device 100 may include an elongate shaft 102 and a tissue collection member 104 disposed on a distal portion of the elongate shaft 102. The tissue collection member 104 may extend along a length of the elongate shaft 102 in the range of about 0.3 inches (7.62 mm) to about 0.55 inches (13.97 mm). The tissue collection device 100 may be configured to be slidably disposed within a lumen of an outer tubular member or sheath. The elongate shaft 102 may extend proximally from a distal end region 106 to a proximal end region (not explicitly shown) configured to remain outside of a patient's body.

During insertion of the tissue collection system into the subject's body, or withdrawal of the system from the subject's body, the tissue collection device 100 may be in a retracted position, with the elongate shaft 102 pulled proximally to position the tissue collection member 104 within a lumen of the outer tubular member. The elongate shaft 102 of the tissue collection device 100 may be pushed distally to move the tissue collection device 100 to an extended position, with the tissue collection member 104 extending out of a distal opening so it is exposed from the outer sheath.

The elongate shaft 102 may include one or more wires twisted into an elongated form. Alternatively, the elongate shaft 102 may be a generally tubular member. In yet another example, the elongate shaft 102 may be a generally solid rod. The elongate shaft 102 may have sufficient flexibility to allow it to bend during insertion of the tissue collection device 100 into or withdrawal of the tissue collection device 100 from the subject's body. The elongate shaft 102 may have sufficient rigidity so that pushing or pulling of the elongate shaft 102 may cause extension or retraction, respectively, of the tissue collection member 104 from the outer tubular member. A proximal end region of the elongate shaft 102 may be gripped by a user such that the user may manually push or pull the elongate shaft 102. Dimensions of the elongate shaft 102 may vary depending upon the subject's anatomy and/or the type of procedure being performed. While not explicitly shown, the elongate shaft 102 may include a distal atraumatic tip. This distal atraumatic tip could be a rounded wire loop, flexible member, a curve flexible member, a bullet shape member, etc. An atraumatic tip may help the shaft to safely navigate to a targeted site.

The tissue collection member 104 may include a plurality of scraping members 108. The tissue collection member 104 may include any number of scraping members 108, such as, but not limited to, one, two, three, four, five, or more. It is contemplated that the scraping members 108 may be formed from metals, metal alloys, polymers, composites, etc. The scraping members 108 may be coupled to the elongate shaft 102 using a number of different techniques, including, but not limited to, welding, soldering, brazing, heat bonding, adhesives, etc. It is contemplated that the coupling technique may vary depending on the material of the elongate shaft 102 and the scraping members 108. In some examples, the elongate shaft 102 and the scraping members 108 may be formed as a single monolithic structure.

The scraping members 108 may be uniformly spaced along the distal end region 106 of the elongate shaft 102 or may be eccentrically spaced, as desired. Each scraping member 108 may extend from a proximal end 110 to a distal end 112. The scraping members 108 may have a truncated funnel shape. For example, the proximal end 110 of each scraping member 108 may have a first cross-sectional dimension and the distal end 112 of each scraping member 108 may have a second cross-sectional dimension smaller than the first cross-sectional dimension. A side wall 114 of each scraping member 108 may be angled from the first cross-sectional dimension to the second cross-sectional dimension. In some embodiments, one or more of the scraping members 108 may include a constant diameter region 116 adjacent to the proximal end 110, although this is not required. It is contemplated that the maximum outer dimension of the scraping members 108 may vary along a length of the tissue collection member 104. For example, it is contemplated that the maximum outer dimension of differing scraping members 108 may reduce in the distal direction to facilitate penetration into a tight stricture while still allowing other scraping members 108 to contact and scrape the stricture.

In some embodiments, the scraping members 108 may each extend about an entirety of the circumference of the elongate shaft 102. In other embodiments, one or more of the scraping members 108 may extend about less than entirety of the circumference of the elongate shaft 102. It is further contemplated that when the scraping members 108 extend about less than entirety of the circumference of the elongate shaft 102, more than one scraping member 108 may be positioned at similar longitudinal locations along the elongate shaft 102, although this is not required.

The proximal ends 110 of the scraping members 108 may be radially spaced from and free from attachment to the elongate shaft 102 to define an open first end while the distal ends 112 of the scraping members 108 may be coupled to or secured to the elongate shaft 102 to define a plurality of cavities 118 between the elongate shaft 102 and an inner surface of the scraping members 108. The tissue collection device 100 may include any number of cavities 118 desired, such as, one, two, three, four, five, or more. It is contemplated that the number of cavities 118 may depend, at least in part, on the number of scraping members 108. The cavities 118 may be configured to trap tissue and/or cells therein. In the illustrated embodiments, the cavities 118 may have a proximally facing opening. However, this is not required. For example, in some embodiments, the proximal end 110 may be affixed to the elongate shaft 102 while the distal end 112 may be radially spaced from the elongate shaft 102. In such a configuration, the distal end 112 may have a greater cross-sectional dimension that the proximal end 110. Such an orientation may reverse the configuration of the cavities 118 such that the cavities have a distally facing opening. It is contemplated that the tissue collection member 104 may include cavities 118 having proximally facing opening, cavities 118 having a distally facing opening, or cavities 118 in which some have a proximally facing openings and some have distally facing openings. When cavities 118 are provided that have differently oriented openings, it is contemplated that the cavities 118 may be arranged in any manner desired. In one example, the proximal most cavities 118 may have proximally facing openings while the distal most cavities 118 may have distally facing openings. This is just one example.

When the tissue collection device 100 is disposed within the outer tubular member, the tissue collection device 100 may be restrained in a compressed reduced diameter or delivery configuration by the outer tubular member surrounding the tissue collection device 100. In the compressed configuration, the tissue collection device 100 may have a smaller diameter than the expanded deployed configuration. The distal end region of the outer tubular member may be positioned such that the outer tubular member surrounds and covers the length of the tissue collection device 100 during delivery. The outer tubular member may have sufficient hoop strength to retain the tissue collection device 100 in its reduced diameter state. However, this is not required. In some cases, the tissue collection device 100 may be free from restraint or compression when disposed within the lumen of the outer tubular member.

To collect a sample, the tissue collection system may be advanced through the body towards the target location, as desired. The tissue collection system may be advanced with or without the use of a guidewire. The tissue collection device 100 may be deployed by actuating the proximal end region of the elongate shaft 102, for example, by distally pushing the proximal end region, while maintaining a first handle coupled to the outer tubular member in a fixed position. Thus, the elongate shaft 102 may be distally advanced relative to the outer tubular member. In other words, the elongate shaft 102 may be distally advanced while the outer tubular member is held stationary. The reverse configuration is also contemplated. For example, the outer tubular member may be proximally retracted while the elongate shaft 102 is held stationary. As the elongate shaft 102 is distally advanced, the biasing force is removed from the exterior of the tissue collection member 104 and the tissue collection member may assume its radially expanded, unbiased, deployed configuration (if the tissue collection member 104 is compressed within the outer tubular member).

Once the tissue collection device 100 is deployed from the outer tubular member, the proximal end region of the elongate shaft 102 may be actuated to repeatedly distally advance, proximally retract, and/or rotate the tissue collection member 104 along the target collection site. This may cause the tissue collection member 104 to brush or scrape against the tissue surface to dislodge and capture cells. Some cells may be trapped within the cavities 118 while others may collect on the surface of the scraping members 108. It is contemplated that the free end (e.g., the proximal end 110 in FIG. 3) may have a sharp edge to scratch or dislodge a sufficient number of cells for analysis.

Once the tissue sample has been collected, the tissue collection system may be removed from the body. In some examples, the elongate shaft 102 and the tissue collection member 104 may be retracted into the outer tubular member for removal. This may help prevent tissue and/or cells from becoming dislodged from the tissue collection member 104 as the tissue collection device 100 is withdrawn from the body.

The elongate shaft 102 may be cut (e.g., using wire cutters or other cutting device) at a location proximal to the tissue collection member 104 such that the tissue collection member 104 may be placed into a sample container. However, this is not required. The sample may be rinsed from the scraping members 108 and/or the cavities 118 using a fluid, such as, but not limited to, saline. It is contemplated that collecting tissue/cells using the scraping members 108 and the cavities 118 may result in a larger sample size than the use of a cytology brush. This may allow the system to collect a sufficient amount of sample for better prediction of biliary cancer (or other ailments).

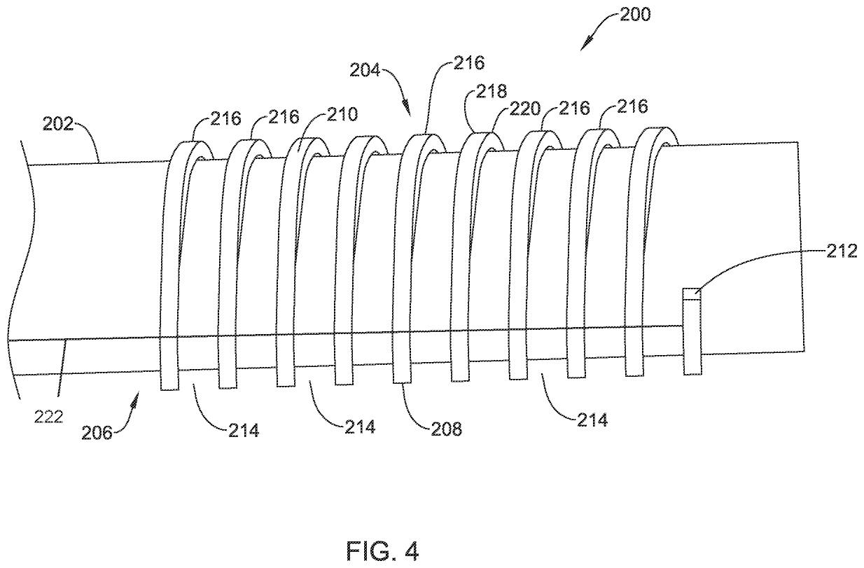

FIG. 4 is a perspective view of distal end region of another illustrative tissue collection device 200. The tissue collection device 200 may be configured to be used with a tissue collection system, such as, but not limited to the tissue collection system 10 described with respect to FIG. 1. For example, the tissue collection device 200 may be used with the system of FIG. 1 in place of tissue collection device 12. Generally, the tissue collection device 200 may include an elongate shaft 202 and a tissue collection member 204 disposed on a distal portion of the elongate shaft 202. The tissue collection member 204 may extend along a length of the elongate shaft 202 in the range of about 0.3 inches (7.62 mm) to about 0.55 inches (13.97 mm). The tissue collection device 200 may be configured to be slidably disposed within a lumen of an outer tubular member or sheath. The elongate shaft 202 may extend proximally from a distal end region 206 to a proximal end region (not explicitly shown) configured to remain outside of a patient's body.

During insertion of the tissue collection system into the subject's body, or withdrawal of the system from the subject's body, the tissue collection device 200 may be in a retracted position, with the elongate shaft 202 pulled proximally to position the tissue collection member 204 within a lumen of the outer tubular member. The elongate shaft 202 of the tissue collection device 200 may be pushed distally to move the tissue collection device 200 to an extended position, with the tissue collection member 204 extending out of a distal opening so it is exposed from the outer sheath.

The elongate shaft 202 may include one or more wires twisted into an elongated form. Alternatively, the elongate shaft 202 may be a generally tubular member. In yet another example, the elongate shaft 202 may be a generally solid rod. The elongate shaft 202 may have sufficient flexibility to allow it to bend during insertion of the tissue collection device 200 into or withdrawal of the tissue collection device 200 from the subject's body. The elongate shaft 202 may have sufficient rigidity so that pushing or pulling of the elongate shaft 202 may cause extension or retraction, respectively, of the tissue collection member 204 from the outer tubular member. A proximal end region of the elongate shaft 202 may be gripped by a user such that the user may manually push or pull the elongate shaft 202. Dimensions of the elongate shaft 202 may vary depending upon the subject's anatomy and/or the type of procedure being performed. While not explicitly shown, the elongate shaft 202 may include a distal atraumatic tip. This distal atraumatic tip could be a rounded wire loop, flexible member, a curve flexible member, a bullet shape member, etc. An atraumatic tip may help the shaft to safely navigate to a targeted site.

In some embodiments, the tissue collection member 204 may include a helically wound coil 208. The helically wound coil 208 may be formed from a wire, filament, or ribbon 210 extending from a proximal end (not explicitly shown) to a distal end 212. While the filament 210 is shown as having a generally rectangular cross-sectional shape, the filament 210 may have other cross-sectional shapes, as desired, including, but not limited to, circular, oblong, polygonal, rectangular, square, etc. It is contemplated that the filament 210 may be formed from metals, metal alloys, polymers, composites, etc.

The filament 210 may be helically wound about the elongate shaft 202 to form the helically wound coil 208 including a plurality of windings 216. The inner surface of the helically wound coil 208 may be positioned adjacent to and coupled to the outer surface of the elongate shaft 202. It is contemplated that an entire length of the helically wound coil 208 need to not be coupled to the elongate shaft 202. It is contemplated that not securing an entirety of the helically wound coil 208 may allow the plurality of windings 216 to actuate to create more space between adjacent windings 216 or compress to reduce the space between adjacent windings 216. The filament 210 may be coupled to the elongate shaft 202 using a number of different techniques, including, but not limited to, welding, soldering, brazing, heat bonding, adhesives, etc. It is contemplated that the coupling technique may vary depending on the material of the elongate shaft 202 and the filament 210. In some examples, the elongate shaft 202 and the helically wound coil 208 may be formed as a single monolithic structure.

The helically wound coil 208 may extend radially beyond an outer surface of the elongate shaft 202 to define a plurality of openings or recesses 214 between adjacent windings 216. The tissue collection device 200 may include any number of recesses 214 desired, such as, but not limited to, one, two, three, four, five, ten, or more. It is contemplated that the number of recesses 214 may depend, at least in part, on a number of windings 216 of the filament 210. The recesses 214 may be configured to trap tissue and/or cells therein. In some embodiments, a thickness of the filament 210 may vary along a length of the helically wound coil 208. For example, the filament 210 may get thinner towards the distal end of the helically wound coil 208, such that the distal end of the helically wound coil 208 extends radially away from the elongate shaft 202 to a lesser extent than the proximal end region. Thus, the recesses 214 adjacent the distal end of the helically wound coil 208 may become shallower. This may allow facilitate penetration into a tight stricture while still allowing portions of the helically wound coil 208 to contact and scrape the stricture

It is further contemplated that the pitch (e.g., distance between adjacent windings 216) of the helically wound coil 208 may varied to vary a size of the recesses 214. In some cases, the helically wound coil 208 may have a uniform pitch along a length thereof. In other cases, the pitch of the helically wound coil 208 may vary or change along a length thereof. For example, the pitch of the helically would coil 208 may be modified to allow for a tight pitch in some areas and more open coil in other areas.

In some embodiments, a distal end region of the helically wound coil 208 may be coupled to a pull wire 222. The pull wire 222 may be proximally retracted to reduce the pitch of the adjacent windings 216 (e.g., bring them closer together) of the helically wound coil 208. This may facilitate proximal retraction of the helically wound coil into the outer tubular member. Releasing the proximal force on the pull wire 222, if so provided, may increase the pitch of the adjacent windings 216 (e.g., move them farther apart) of the helically wound coil 208 to allow for more tissue penetration between the windings 216. In some cases, a distal pushing force may be used to reduce the pitch of adjacent windings 216. For example, if the pull wire 222 is coupled to proximal end region of the helically wound coil 208, a distal pushing force may collapse or reduce the pitch of the windings 216.

When the tissue collection device 200 is disposed within the outer tubular member, the tissue collection device 200 may be restrained in a compressed reduced diameter or delivery configuration by the outer tubular member surrounding the tissue collection device 200. In the compressed configuration, the tissue collection device 200 may have a smaller diameter than the expanded deployed configuration. The distal end region of the outer tubular member may be positioned such that the outer tubular member surrounds and covers the length of the tissue collection device 200 during delivery. The outer tubular member may have sufficient hoop strength to retain the tissue collection device 200 in its reduced diameter state. However, this is not required. In some cases, the tissue collection device 200 may be free from restraint or compression when disposed within the lumen of the outer tubular member.

To collect a sample, the tissue collection system may be advanced through the body towards the target location, as desired. The tissue collection system may be advanced with or without the use of a guidewire. The tissue collection device 200 may be deployed by actuating the proximal end region of the elongate shaft 202, for example, by distally pushing the proximal end region, while maintaining a first handle coupled to the outer tubular member in a fixed position. Thus, the elongate shaft 202 may be distally advanced relative to the outer tubular member. In other words, the elongate shaft 202 may be distally advanced while the outer tubular member is held stationary. The reverse configuration is also contemplated. For example, the outer tubular member may be proximally retracted while the elongate shaft 202 is held stationary. As the elongate shaft 202 is distally advanced, the biasing force is removed from the exterior of the tissue collection member 204 and the tissue collection member may assume its radially expanded, unbiased, deployed configuration (if the tissue collection member 204 is compressed within the outer tubular member).

Once the tissue collection device 200 is deployed from the outer tubular member, the proximal end region of the elongate shaft 202 may be actuated to repeatedly distally advance, proximally retract, and/or rotate the tissue collection member 204 along the target collection site. This may cause the tissue collection member 204 to brush or scrape against the tissue surface to dislodge and capture cells. Some cells may be trapped within the recesses 214 while others may collect on the surfaces or edges of the helically wound coil 208. It is contemplated that the edges 218, 220 of the filament 210 may have a sharp edge to scratch or dislodge a sufficient number of cells for analysis. For example, an outer surface of the filament 210 may be flat to create sharp edges 218, 220 which are configured to scrape stricture cells, including stiff stricture cells.

Once the tissue sample has been collected, the tissue collection system may be removed from the body. In some examples, the elongate shaft 202 and the tissue collection member 204 may be retracted into the outer tubular member for removal. This may help prevent tissue and/or cells from becoming dislodged from the tissue collection member 204 as the tissue collection device 200 is withdrawn from the body.

The elongate shaft 202 may be cut (e.g., using wire cutters or other cutting device) at a location proximal to the tissue collection member 204 such that the tissue collection member 204 may be placed into a sample container. However, this is not required. The sample may be rinsed from the helically wound coil 208 and/or the recesses 214 using a fluid, such as, but not limited to, saline. It is contemplated that collecting tissue/cells using the helically wound coil 208 and the recesses 214 may result in a larger sample size than the use of a cytology brush. This may allow the system to collect a sufficient amount of sample for better prediction of biliary cancer (or other ailments).

The materials that can be used for the various components of the medical device system 10 (and/or other systems disclosed herein) and the various elements thereof disclosed herein may include those commonly associated with medical devices. For simplicity purposes, the following discussion refers to the tissue collection system 10, 100, 200 and/or the tissue collection member 22, 104, 204. However, this is not intended to limit the devices and methods described herein, as the discussion may be applied to other elements, members, components, or devices disclosed herein, such as, but not limited to, the outer tubular members 18, handle 30, elongate shafts 20, 102, 202, etc. and/or elements or components thereof.

In some embodiments, the tissue collection system 10, 100, 200, the tissue collection member 22, 104, 204, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, Marl MARLEX®ex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of tissue collection system 10, 100, 200, the tissue collection member 22, 104, 204, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the tissue collection system 10, 100, 200 and/or the tissue collection member 22, 104, 204 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the tissue collection system 10, 100, 200 and/or the tissue collection member 22, 104, 204 to achieve the same result.

In some embodiments, a degree of Magnetic Resonance Imaging (MRI) compatibility is imparted into tissue collection system 10, 100, 200 and/or the tissue collection member 22, 104, 204. For example, the tissue collection system 10, 100, 200 and/or the tissue collection member 22, 104, 204, and/or components or portions thereof, may be made of a material that does not substantially distort the image and create substantial artifacts (e.g., gaps in the image). Certain ferromagnetic materials, for example, may not be suitable because they may create artifacts in an MRI image. The tissue collection system 10, 100, 200 and/or the tissue collection member 22, 104, 204, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, an exterior surface of the medical device system 10, 100, 200 (including, for example, an exterior surface of the delivery system) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the outer sheath, or in embodiments without an outer sheath over portions of the delivery system, or other portions of the medical device system 10, 100, 200. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinyl alcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A tissue collection system, the tissue collection system comprising:
a tubular member having a proximal end region and a distal end region and defining a lumen extending from the proximal end region to the distal end region;
an elongate shaft slidably disposed within the lumen of the tubular member, the elongate shaft extending from a proximal end region to a distal end region; and
a tissue collection member disposed adjacent to the distal end region, the tissue collection member comprising a helically wound coil including a plurality of windings and a plurality of recesses between adjacent windings of the plurality of windings.

2. The tissue collection system of claim 1, wherein the tissue collection member is coupled to the elongate shaft.

3. The tissue collection system of claim 1, wherein the tissue collection member is coupled to the elongate shaft along less than an entire length of the tissue collection member.

4. The tissue collection system of claim 1, further comprising a pull wire coupled to the helically wound coil.

5. The tissue collection system of claim 4, wherein the pull wire is configured to be actuated to adjust a pitch of the helically wound coil.

6. The tissue collection system of claim 1, wherein an inner surface of the helically wound coil contacts an outer surface of the elongate shaft.

7. The tissue collection system of claim 1, wherein the helically wound coil is formed from a filament having a thickness.

8. The tissue collection system of claim 7, wherein the thickness of the filament reduces from a proximal end to a distal end thereof.

9. A tissue collection system, the tissue collection system comprising:
a tubular member having a proximal end region and a distal end region and defining a lumen extending from the proximal end region to the distal end region;
an elongate shaft slidably disposed within the lumen of the tubular member, the elongate shaft extending from a proximal end region to a distal end region; and
a tissue collection member disposed adjacent to the distal end region, the tissue collection member comprising a ribbon having a proximal end, a distal end, a first lateral side extending between the proximal end and the distal end, and a second lateral side extending between the proximal end and the distal end, the ribbon wound about the elongate shaft to form a helically wound coil including a plurality of windings.

10. The tissue collection system of claim 9, wherein the first lateral side of the ribbon is adjacent to the elongate shaft and the second lateral side of the ribbon is radially spaced from the elongate shaft.

11. The tissue collection system of claim 10, further comprising a plurality of cavities, the plurality of cavities defined by a space between an inner surface of the ribbon and the elongate shaft.

12. The tissue collection system of claim 11, wherein the plurality of cavities each have an opening.

13. The tissue collection system of claim 12, wherein each opening of the plurality of cavities is distal facing.

14. The tissue collection system of claim 12, wherein each opening of the plurality of cavities is proximal facing.

15. The tissue collection system of claim 9, wherein a maximum outer diameter of the tissue collection member reduces in diameter in a distal direction.

16. A tissue collection system, the tissue collection system comprising:
a tubular member having a proximal end region and a distal end region and defining a lumen extending from the proximal end region to the distal end region;
an elongate shaft slidably disposed within the lumen of the tubular member, the elongate shaft extending from a proximal end region to a distal end region; and
a tissue collection member disposed adjacent to the distal end region, the tissue collection member comprising a plurality of scraping members having an open first end and a second end coupled to the elongate shaft.

17. The tissue collection system of claim 16, wherein each scraping member of the plurality of scraping members defines a cavity therein.

18. The tissue collection system of claim 16, wherein the open first end of the plurality of scraping members is distally facing.

19. The tissue collection system of claim 16, wherein the open first end of the plurality of scraping members is proximally facing.

20. The tissue collection system of claim 16, wherein a maximum outer diameter of the tissue collection member reduces in diameter in a distal direction.
